(19) United States
(12) Patent Application Publication
(10) Pub. No.: US 2018/0207328 A1
(43) Pub. Date: Jul. 26, 2018

(54) Implantable Compositions for Pain Management

(71) Applicant: Kamalesh K. Rao, South San Francisco, CA (US)

(72) Inventor: Kamalesh K. Rao, South San Francisco, CA (US)

(21) Appl. No.: 15/876,221

(22) Filed: Jan. 22, 2018

Related U.S. Application Data
(60) Provisional application No. 62/449,102, filed on Jan. 22, 2017.

(51) Int. Cl. A61L 31/16; A61K 31/196; A61L 31/14; A61K 47/14; A61K 47/10; A61K 47/32; A61P 25/04; A61L 31/04; A61K 31/192; A61K 31/407

(52) U.S. Cl. CPC A61L 31/16; A61K 31/196; A61L 31/148; A61K 47/14; A61K 47/10; A61L 2300/402; A61P 25/04; A61L 31/048; A61K 31/192; A61K 31/407; A61K 47/32

(57) ABSTRACT

The invention includes an implantable composition for management of pain in animals. The composition includes one or more active ingredients, a biocompatible polymer based drug delivery matrix, and other compounds to facilitate composition manufacture and durability. The composition disclosed herein is administered as a veterinary implant device. The implant is inserted subcutaneously into a patient and is designed to function in vivo for prolonged periods of time ranging from multiple weeks to months or even years. Accordingly, the composition must provide a constant release of one or more active ingredients over an extended period of time. To enable combination therapy for pain management, the composition may also include one or more layers of active ingredients seeded into one or more drug delivery systems. Multiple compositions having one or more layers loaded with different active ingredients may also be used in combination therapies.

### CROSS REFERENCE TO RELATED APPLICATION

This application claims priority to U.S. Provisional Patent Application No. 62/449,102 filed Jan. 22, 2017, which is incorporated herein by reference in its entirety.

### FIELD OF INVENTION

The invention comprises compositions for implantable drug delivery systems that dispense a wide variety of pain management drugs used to treat chronic pain. More particularity, the disclosure relates to pharmaceutical compositions including one or more active ingredients, drug delivery systems, and other compounds for formation stability and dose manufacturing.

### BACKGROUND

Medication of pets and companion animals is an important issue in the developed world. Pets play a crucial role in the physical and emotional well being of humans. Accordingly, demand for providing pets with healthcare and medicines on par with human care is increasing.

Accompanying the rise in demand, an aging pet population further exacerbates the need for sophisticated pet medical care. Food, nutrition, and healthcare contribute to longevity of animals. Modern improvements in these areas have produced a growing population of older pets and a corresponding increase in age-related joint (arthritis and osteoarthritis), coronary (cardiovascular), hypertension, cognitive, and immune-system-related conditions as well as diabetes and cancer. More pets in need of healthcare results in prescribing more medication along with an additional increase in supervision and monitoring care needed to administer the medication. Older pets also often have stronger bonds with their owners. Increased loyalty and affection for their pet companions drives pet owners to have an even greater willingness to invest in state of the art treatment options for their pets further fortifying demand for advanced veterinary medicine.

Currently, the majority of pet medications are dispensed orally. For pets, oral drug delivery systems are notoriously unreliable because swallowing pills frequently makes pets uncomfortable. Pets are also often very good at detecting pills in their food and proficient at hiding small pills in their lips, gums, or between their teeth. Accordingly, there is no guarantee that medicines dispensed orally will be swallowed by a pet. To ensure more medicines are ingested by animals, current oral modes of drug delivery require careful supervision by the pet owner. A large percentage of pet owners, however, are sick, disabled, or elderly and therefore either unable or unwilling to administer medicines to their pets as prescribed. In short, oral drug delivery systems are not a good fit for pets because they are frustrating to animal owners, cause stress to the animals, and contribute to non-compliance, lower quality, and increased cost of veterinary care.

Many current innovations in veterinary medicine seek to increase companion animal healthcare compliance. As used herein the term “compliance” refers to the willingness to follow a prescribed course of treatment. Compliance can be improved by reducing treatment frequency, making treatment easier to administer and remember, and by avoiding follow-up treatments to resolve the same condition. Technical innovations can be developed to facilitate each of these advances. For example, one or more active ingredients for treating the same or unrelated condition can be combined in a combination therapy to reduce the number of medications that have to be administered. New, improved, and superior drug delivery systems can also be developed to reduce reliance on oral drug delivery. Additionally, electronic treatment scheduling and reminder systems can help pet owners develop a routine for medicating their animal companions.

Previous work on drug delivery systems has produced implantable delivery systems with a drug or a combination of drugs encapsulated in a polymeric matrix. These systems use a drug delivery matrix composed of a single polymer or a mixture of polymers, with or without excipients, to provide long-term release of active ingredients. Polymers used in state of the art drug delivery matrices may be composed of a biocompatible polymer that is either biodegradable or non-biodegradable. Although previous drug delivery innovations have produced useful alternatives to orally dispensed treatments, none of the inventions have specifically addressed the following problems:
Non-compliance in the field of veterinary medicine caused by high dosing frequency;
Non-compliance in the field of veterinary medicine caused by required follow-up treatments due to the lack of usable combination drug therapy;
Non-compliance in the field of veterinary medicine caused by the need for human supervision when administering medicines to animals;
Low quality of care and high cost of treatment due to undesirable side effects such as gastrointestinal distress, and liver and kidney toxicity; and
Low pet quality of life as a result of non-effective methods of pre-emptive analgesia—the administration of analgesic drugs to prevent pain in advance of pain stimulus occurring.

The object of the present invention is to simultaneously solve the above mentioned problems of non-compliance, low quality of care, high cost of treatment, low quality of pet life, and better methods for pre-emptive analgesia by providing compositions for implantable drug delivery systems that dispense a wide variety of pain management drugs used to treat chronic pain.

### SUMMARY OF INVENTION

The compositions described herein provide superior, reliable, compliant, and safe drug delivery platforms, relative to existing alternatives. The compositions provide implantable drug delivery systems that do not require supervision by the pet owner or a veterinarian. By delivering assurance that pets are receiving medicines continuously at the prescribed dosage with no monitoring, the drug delivery system contributes to pet owner emotional well-being. The system also helps pets by providing relief from forceful oral drug delivery, minimizing or eliminating adverse effects such as GI distress and liver toxicity while also reducing the potential for tampering and abuse. The compositions of the present invention improve compliance, provide comfort, eliminate anxiety, and speed up recovery.

Pre-emptive analgesia is an antinociceptive treatment that prevents establishment of altered processing of afferent input, which amplifies postoperative pain. Frequently methods of preemptive analgesia combine different classes of analgesic drugs to effectively manage pain and limit side effects. In an approach called multimodal analgesia, drugs such as Nonsteroidal Anti-Inflammatory Drugs (NSAID's), opioids, N-Methyl-D-Aspartate (NMDA) antagonists, local anesthetics, and α-2 adrenoceptor agonists are used in combination to achieve optimal results.

Compositions of the present invention are useful in pre-emptive analgesia techniques because these compositions support the release of multiple active ingredients from the same drug delivery system. Active ingredients included in the compositions contemplated by this invention include any species of the group comprising, NSAID's with opioids, NSAIDS's with local anesthetics, NSAIDS's with α-2 adrenoceptor agonists, NSAIDS's with NMDA antagonists, NSAIDS's with α-2 adrenoceptor agonists, Carprofen, Carprofen with morphine, Carprofen with Amantadine, Carprofen with Gabapentin, Carprofen with Tramadol, Meloxicam with Gabapentin, Meloxicam, Meloxicam with Morphine, Meloxicam with Amantadine, Tramadol with Gabapentin, Tramadol with Amantadine, Tramadol with Amitriptyline, Dexmedetomidine with Morphine, Xylazine with Morphine, Dexmedetomidine with Remifentanil, Dexmedetomidine with Fentanyl, Amantadine and Amitriptyline combination, Amantadine with Morphine, Amantadine, Dexmedetomidine with morphine, Dexmedetomidine with Fentanyl, Dexmedetomidine with Buprenorphine, Dexmedetomidine with Ketamine, Xylazine with Ketamine, Xylazine with Morphine, Xylazine in combination with Fentanyl, Xylazine with Buprenorphine, Cinchophen (NSAID) and prednisolone (corticosteroid), Duloxetin, Pamidronate, Zoledronate, Morphine with benzodiazepines, Morphine with acepromazine, Morphine with Romifidine, Morphine with Xylazine, Morphine with Medetomidine, Morphine with Dexmedetomidine, Fentanyl with benzodiazepines, Fentanyl with Xylazine, Fentanyl with Dexmedetomidine, Fentanyl with Medetomidine, Fentanyl with Romifidine, Fentanyl with acepromazine, Buprenorphine with Medetomidine, Buprenorphine with Dexmedetomidine, Buprenorphine with Romifidine, Buprenorphine with Xylazine, Buprenorphine with benzodiazepines, Buprenorphine with acepromazine, and Pamidronate with zoledronate.

An example composition includes about 5 to 85% total active ingredient. In embodiments comprising more than one active ingredient, actives may be included in the same or different amounts. Various embodiments with two active ingredients include 5 to 60% active ingredient A and 5 to 60% active ingredient B. For example, one compositions includes a polymer drug delivery matrix loaded with 20% Ibuprofen and 20% Ketorolac.

The above active ingredients may be combined within a multilayered drug delivery system to provide consistent dosage of one or more active ingredients over an extended period of time. Compositions of the present invention release one or more active ingredients for pre-emptive analgesia and other pain management techniques continuously in vivo for at least about 1, 3, 9, 12, 15, 18, 21, or 24 months.

Drug delivery systems included in the compositions described herein include a drug delivery matrix comprised of one or more chemicals selected from the group comprising: Hydroxypropyl cellulose, Glyceryl behenate, Ethyl-prop-2-enoate, Ethyl-vinyl-acetate, Polyethylene glycol, Glycerin triacetate, and Triethyl citrate.

To make the compositions of the present invention the above chemicals may be polymerized and extruded to produce polymers selected from the group comprising: HPC-EF, HPC-ELF, Eudragit RSPO, EVA VA 800, PEG 600. Plasticizers such as triacetin, and triethyl citrate may also be added to compositions to make manufacturing easier and more efficient.

Compositions may also be coated with a degradable coating to further regulate drug release. In various examples, a lipid excipient comprising esters of behenic acid and glycerol (Compritol 888 ATO) is applied to the external surface of the composition to prevent the release of an initial burst of active ingredient shortly after the composition is implanted in a patient. In various embodiments, compositions of the present invention include 5 to 20% Compritol 888 ATO.

In various embodiments, compositions of the present invention include 5 to 80% drug delivery polymer. The drug delivery polymer may include one or more biocompatible, nonerodible polymers for formation of the drug delivery matrix. In various examples, a composition may include 5 to 50% HPC-ELF, 5 to 30% HPC-HF, 5 to 90% Eudragit, and/or 5 to 90% EVA. In examples with EVA, the vinyl acetate content is often about 10 to 40%, more often 20 to 30%, and most often 22 to 28%. In some embodiments the vinyl acetate content is 28%.

### BRIEF DESCRIPTION OF DRAWINGS

FIG. 1 illustrates an example embodiment of a coaxial implant composition described herein.

FIG. 2 illustrates cumulative drug release for two implantable compositions including EVA and loaded with 20% Ibuprofen.

FIG. 3 illustrates percent cumulative drug release for two implantable compositions including EVA and loaded with 20% Ibuprofen.

FIG. 4. Illustrates cumulative drug release for two implantable compositions including EVA and loaded with 20% Ketorolac tromethamine.

FIG. 5 illustrates percent cumulative drug release for two implantable compositions including EVA and loaded with 20% Ketorolac tromethamine.

### DETAILED DESCRIPTION

The compositions described herein comprise two major components—a drug delivery matrix that acts as a reservoir to hold the active ingredient and the active ingredient itself. In some examples the drug delivery matrix is a synthetic polymer, for example, EVA. Other examples, have drug delivery matrices comprised of biopolymers such as collagen. Composition of the present invention may also include one or more excipients such as surfactants, lubricants, and adjuvants. These compounds are mixed with the active ingredient-polymer mixture to facilitate release of one or more active ingredients in a controlled manner at desired therapeutic levels. To make the composition, the active ingredient is mixed with the polymer using any method, known to a person skilled in the art. The composition is then manufactured as an implant of any suitable shape as necessary for the application.

In one example, implants comprising the compositions of the present invention are manufactured using hot melt extrusion (HME). HME is a promising technology for manufacturing medical implants that involves application of heat, pressure, and agitation through an extrusion channel to mix materials together, then force them out through a die. It blends materials while imparting high shear stress to break-up particles and disperse them. It offers several advantages over conventional manufacturing techniques including
Continuous process;
High throughput;
Solvent-free technique;
Increased solubility and bioavailability of poorly water-soluble drugs;
No downstream processing;
Compatibility with low compressibility index Active Pharmaceutical Ingredients (API); and
Reduced exposure to oxygen in extrusion channel.

Implantable compositions produced by HME typically have cylindrical forms. Compositions are cut to desired length and have dimensions of about 1 to about 4 mm in diameter and about 1 to 5 cm in length. In one example, compositions of the present invention are extruded through a die with dimensions of about 1.5 to 2.75 mm. The example manufacturing parameters are for descriptive purposes only. Other composition shapes and sizes and manufacturing techniques are contemplated and are within the skill of the art.

The release kinetics of an active ingredient from a polymeric drug delivery system are a function of the active's molecular weight, lipid solubility, and charge. Release kinetics further depend on the characteristics of the drug delivery matrix including percentage drug loading, excipients, and matrix coating. These factors are weighed in designing compositions of the present invention. For example, active ingredients with excellent water solubility including hydromorphone give a favorable release pattern from ethylene vinyl acetate (EVA) based matrices. Additional factors including drug specific pharmacology and toxicology as well as therapeutic goals are also incorporated into the rationale for compositions described herein. Duration of the active ingredient release period and the desired plasma levels for different actives form additional design considerations.

Variation in thickness and diameter of implantable compositions and the number of implants used in a patient provide flexibility in the amount of active ingredient released per hour. The optimal drug dosage rate dispensed by the implant depends on factors including the condition for which the “active ingredient” is being administered, the physiology of the subject, species in which it is used, and body weight of the patient. The dosage rate will be readily ascertainable to veterinarians, physicians, and other medical experts. For treatment of pain, the analgesic “active ingredient” is desirably released at a rate that maintains plasma levels of the active ingredient at a therapeutically effective level. Moreover, depending upon the nature of the pain condition being treated and the particular subject, implants including compositions of the present invention may be used to release therapeutically effective amounts of one or more active ingredients for several weeks, months, or even up to two years or longer. As used herein, “therapeutically effective amount” or “therapeutically effective level” refers to the amount of active ingredient required to render a desired therapeutic outcome (i.e., reduction of self-administration of nonprescribed active ingredients, or analgesic relief of pain).

After subcutaneous implantation in an animal, a composition of the invention releases one or more active ingredients for pre-emptive analgesia and other pain management therapies continuously in vivo at a rate that results in a plasma level of at least about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 5, or 10 ng/ml. Drugs are released from compositions through a network of channels extending from the core to the surface of the drug delivery matrix.

In various embodiments, compositions of the present invention release one or more active ingredients for pain management therapies at a rate of at least about 0.01, 0.05, 0.1, 0.5, 1, 2, 3, 4, 5, 6, 7, 8, 9, 10, 20, 30, 50, or 100 mg/day in vitro or in vivo. Two example embodiments described below release about 0.5 mg/day and about 0.02 mg/day in vitro. In various other embodiments, compositions release active ingredients for treatment of chronic pain at a rate of at least about 10 to about 1000 ppm/day steady state in vivo or in vitro.

Embodiments of the present invention include implantable compositions for combination therapy. As used herein, “combination therapy” refers to treatment methods that administer two or more active ingredients. Combination therapies may be used to treat one or more conditions and therapies for treating two or more conditions may target related or unrelated conditions. One example implantable composition including two or more active ingredients comprises a single core or compartment for disposing two or more active ingredients. To provide different release profiles, one or more active ingredients may be coated with a bio-erodible or biodegradable polymer. Two or more coatings may be applied to the same active ingredient and the degradation rates of the coatings may be varied to provide faster or slower release rates.

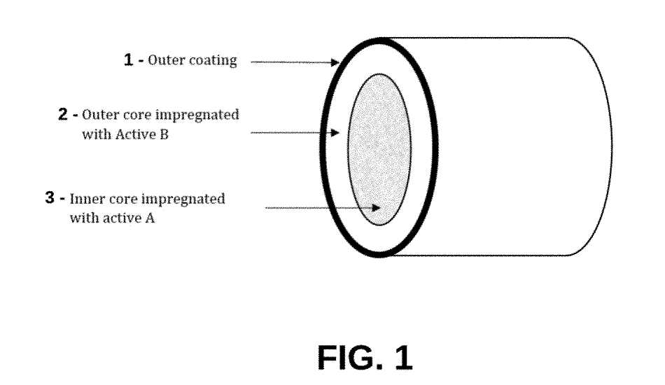

Another example composition includes two layers for disposing one or more active ingredients. FIG. 1 illustrates an example multi-layer embodiment having two coaxial cores. The inner core 3 and outer core 2 include one or more active ingredients and a drug delivery matrix. The composition further includes at least one outer coating 1 on the external surface of the implant. A second outer coating may be applied to the outside of the inner core 3 to separating the inner and outer layers.

In this example, the inner core 3 and outer core 2 are concentric layers comprising drug delivery systems made of one or more polymer materials. The material composition of each concentric layer may include the same polymer with different layers having different concentrations, monomer ratios, or degree of crosslinking. Alternatively, the concentric layers may further include one or more different polymers for each layer with different layers having different compositions or the same compositions with different concentrations, monomer ratios, or degree of crosslinking.

Active ingredients are loaded into the drug delivery matrix of each concentric layer. In this example, the active ingredient loaded into the inner core 3 is different from the active ingredient loaded into the outer core 2. This arrangement is ideal for combination treatments requiring two or more active ingredients. Other examples include the same active ingredient in each layer or two or more active ingredients in a single layer. In embodiments having two or more active ingredients in one layer, one or more of the active ingredients may be coated with a non-erodible or biodegradable polymer to regulate the rate of drug release.

Outer coatings may be added to the external surface of one or more concentric layers. In one example, the implantable composition comprises an outer coating 1 around the external surface of the outer core 2. The outer coating helps regulate drug release by preventing an initial burst of the active ingredient immediately after the implant is inserted into a patient. In this example the outer coating encloses the outer surface of the implant composition in a layer of bio-erodible polymer. Once the implant has established equilibrium in vivo shortly after surgery, the outer coating erodes to dispense drugs through pores on the external surface of the implant. The delay provided by the outer coating keeps the release of drugs constant for the full life of the implant and reduces risk of side effects associated with elevated plasma levels of one or more active ingredients including liver and kidney damage, upset stomach, and loss of appetite. The length of the delay provided by the outer coating may be varied by changing the composition of the outer coating. Coatings comprising more robust polymers may provide a delay of several days or weeks. Alternatively, readily degradable coatings may only provide a few minutes or hours of delay.

In various examples, the outer coating 1 comprises a lipid excipient including esters of behenic acid and glycerol. In one example, Compritol 888 ATO is applied to the external surface of the composition to prevent the release of an initial burst of active ingredient shortly after the composition is implanted in a patient. In various embodiments, compositions of the present invention include 5 to 20% Compritol 888 ATO.

Currently available, biodegradable polymer drug delivery matrices for long-term delivery of analgesics suffer from inability to maintain a constant drug release rate for several months or longer time periods because channels form in the polymer matrix as it degrades, resulting in increased release rates. In addition to channeling, other durability issues limiting state of the art biodegradable implants include softening over time and gradual loss of structural integrity. The tendency of implants to fragment makes implant removal difficult if not impossible. The drug delivery matrix of the present invention overcomes the disadvantages offered by conventional biodegradable polymer matrices.

Compositions of the present invention include a drug delivery matrix comprising a biocompatible, non-erodible polymer that exhibits generally linear in vivo release kinetics for a wide variety of active ingredients. As used herein, “non-erodible or non-biodegradable matrix” refers to a polymer drug delivery matrix that is resistant to chemical and/or physical destruction by the environment of use (the body of an animal patient), such that the matrix remains essentially intact throughout the release period. In this instance, “intact” means that the implant retains it's physical properties for the duration of use in the use environment, remains stable and retains full functionality after extended time in storage before use, and does not crumble or fragment during use. EVA and silicone are two polymer materials used in compositions described herein. These polymers possess all of the properties mentioned above and are familiar to regulatory agencies such as the FDA.

Compositions of the instant invention are suitable for sustained release of the “active ingredient” for treatment of chronic pain in animals. As used herein, “sustained release” refers to the release of an active ingredient such that the blood concentration of the active is kept within a therapeutic range for an extended duration without crossing the toxicity threshold. Implants incorporating compositions of the present invention generally exhibit near zero-order pharmacokinetics in vivo. Drugs are delivered to the patient with kinetics similar to an Intravenous (IV) drip. Unlike IV techniques, however, the drug delivery system described herein does not require external medical equipment or expert personnel. Generally, after implantation, devices release therapeutically effective amounts of the active ingredient for periods of several months up to one year or longer.

Compositions of the present invention include one or more active ingredients for relieving pain. Active ingredients selected for use in example compositions include NSAIDS, steroidal active ingredients, Disease Modifying Antirheumatic Drugs (DMARDS), opioids, α-2 adrenergic agonists, Tricyclic antidepressants (TCA's), Serotonin and Norepinephrin Reuptake Inhibitors (SS(N)RI's), bisphosphonates, and Polysulfated Glycosaminoglycan's (PSGAG's), analgesics, and other anaesthetics incorporated (encapsulated or loaded onto) into a polymeric, non-erodible matrix. As used herein, “active ingredient” refers to free base, free acid, and pharmaceutically acceptable salts thereof, of the active ingredient in use.

In compositions of the present invention, active ingredients are combined with a non-erodible polymeric matrix that remains intact in vivo for extended periods of time, typically months or years. When implanted subcutaneously, implants incorporating compositions of the present invention continuously release the “active ingredient” for a prolonged duration with a pseudo or near zero order release rate. Incorporating one or more active ingredients into the polymeric matrix results in disruption of the homogeneity of the matrix. The disruption forms a series of interconnecting channels and pores throughout the body of the matrix. These complex networks extend from the center of matrix out to its outer surface thereby creating pathways for drugs to travel out of the matrix and enabling release of the active ingredient from the implant surface. Active ingredient molecules encapsulated in the matrix are released in a sustained and predictable manner over time via diffusion through the network of channels and pores.

The release rate can be altered by modifying the percent active ingredient loading, porosity of the matrix, structure of the implantable device, and hydrophobicity of the matrix; applying a biodegradable coating to a layer of the non-erodible matrix; incorporating an active ingredient adsorbed on a resin via ionic bonding, hydrogen bonding, Van der Waals attraction, hydrophobic interaction and/or covalent bonding; adding of excipients such as surfactants to the polymer matrix to manipulate the release rate of the active ingredient; or adding a hydrophobic coating to the exterior of the implantable device.

Ethylene vinyl acetate copolymer (EVA) is one material used in the drug delivery matrix of the implantable compositions described herein. Other non-erodible materials may be used, for example, silicone, hydrogels such as crosslinked poly(vinyl alcohol) and poly(hydroxy ethylmethacrylate), acyl substituted cellulose acetates and alkyl derivatives thereof, partially and completely hydrolyzed alkylene-vinyl acetate copolymers, unplasticized polyvinyl chloride, crosslinked homo- and copolymers of polyvinyl acetate, crosslinked polyesters of acrylic acid and/or methacrylic acid, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, and chlorosulphonated polyolefines, and combinations thereof. Biopolymers such as collagen may also be incorporated into drug delivery matrices of the present invention.

### EXAMPLES

The following examples are intended to illustrate but not limit the invention.

### Example 1: Materials

The following materials were used: hydroxypropyl cellulose, glyceryl behenate, ethyl prop-2-enoate, ethyl vinyl acetate, polyethylene glycol, glycerin triacetate, triethyl citrate. Table 1 below displays some physical properties of selected drug delivery materials.

TABLE 1
Drug Delivery Materials

| Materials | Properties | | |
|---|---|---|---|
| (Supplier) | Glass Transition (Tg) | Toxiticty | Biodegradable |
| HPC-EF | 120° C. | LD₅₀: 0.25 g/kg | Yes |
| (Ashland) | | | |
| HPC-ELF | 120° C. | LD₅₀: 0.25 g/kg | Yes |
| (Ashland) | | | |
| Compritol | 70° C. | LD₅₀: 5 g/kg | Yes |
| (Gattefosse) | | | |
| Eudragit | 64° C. | — | No |
| (Evonik) | | | |
| EVA | 35-40° C. | Not toxic and | No |
| (Amizara) | | Non-irritant | |

### Example 2: Preparation of Implantable Compositions

Compositions were prepared using an HME technique performed with a single screw extruder device. To verify extrudablility of the drug delivery materials, implantable rods were extruded with and without any active ingredients (AIs). Table 2 below displays the extrusion parameters for placebo and AI loaded implantable rods. All drug delivery materials with the exception of Compritol had excellent extrudability with a wide range of AIs.

TABLE 2
Extrusion Parameters

| | Properties | | |
|---|---|---|---|
| Active Ingredient | Screw | | |
| (Supplier) | Speed | Temperature | Die Diameter |
| Placebo | 40 RPM | 85° C. | 2.58 mm |
| Ibuprofen | 30 RPM | 80° C. | 2.58 mm |
| (Chemicals and Pharmas. Ltd.) | | | |
| Ketorolac tromethamine | 40 RPM | 90° C. | 2.58 mm |
| (MSN Laboratories Pvt. Ltd.) | | | |

To prepare samples for extrusion, the required quantity of drug delivery materials and AIs were weighed. Compounds were then uniformly mixed with a small amount (1 to 20%) plasticizer, for example, polyethylene glycol (PEG), triacetin, or triethyl citrate (TEC). Implantable compositions were then extruded and cut to the desired length (2 to 5 cm).

### Example 3: In Vitro Characterization of Implantable Compositions

Compositions prepared as described above were characterized for rate of drug release. Implantable compositions included EVA VA 800 grade ethylene vinyl acetate containing 28% vinyl acetate. Compositions were loaded with 20% active ingredient to study the release kinetics. For this example, two EVA drug delivery systems loaded with 20% Ibuprofen and two EVA drug delivery systems loaded with 20% Ketorolac tromethamine were extruded. Extruded compositions resembled cylindrical rods and were cut to 5 cm in length.

To test release kinetics, extruded rods were weighed and placed in 10 ml of 7.4 pH buffer solution disposed in a bath shaker. Solutions were maintained at a constant temperature of 37° C. and intermediately shaken. The pH, temperature, and intermediate agitation of solutions imitate in vivo conditions, such as, inside the body cavity of a mammal. Solutions were sampled by withdrawing 4 ml aliquots of buffer solution and replacing the same amount with fresh buffer. Aliquot samples were analyzed using UV visible spectroscopy performed on a Labman Scientific UV Visible spectrophotometer. On this instrument, Ibuprofen was detected at a wavelength of 232 nm and Ketorolac tromethamine was detected at 322 nm. The calibration curve for determining the concentration of the active ingredients indicated linearity for Ibuprofen over a range of 5 to 35 micrograms (mcg) and linearity for Ketorolac tromethamine over a range of 4 to 14 mcg. To enhance compatibility with instrumentation selected for analysis, samples to be analyzed were diluted such that the absorbance lies in the range of 0.2 to 0.8. The required dilution factor was later multiplied to obtain the concentration of active ingredient present in the sample.

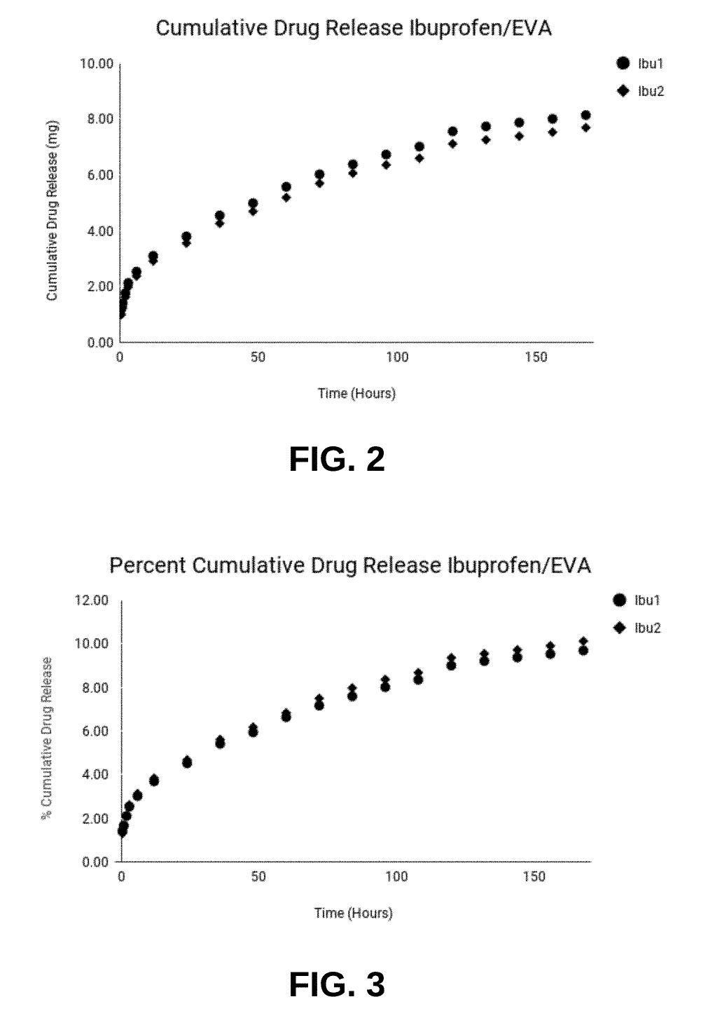

FIG. 2 shows the release of Ibuprofen from two implantable compositions loaded with 20% Ibuprofen. FIG. 3 shows the percent cumulative drug release for both compositions. The in vitro release data indicates a high amount of drug release for both compositions in the first half hour. Compositions reached steady state after 1 hour and maintained steady state between 1 and 168 hours. Over the 1 week trial period, compositions released 8.13 mg and 7.69 mg of Ibuprofen. The percent cumulative drug release for Ibu/EVA compositions was 9.69% and 10.11%.

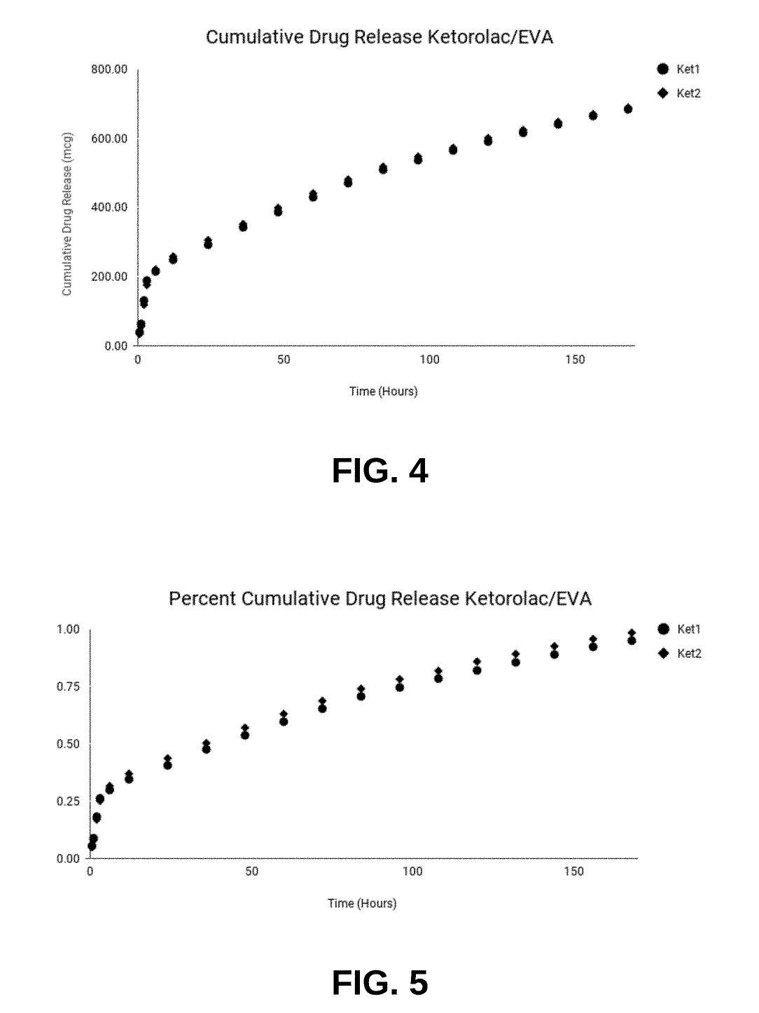

FIG. 4 shows the release of Ketorolac tromethamine from two implantable compositions loaded with 20% Ketorolace tromethamine. FIG. 5 shows the cumulative percent drug release for both compositions. As with the Ibu/EVA compositions, the in vitro release data indicates a high amount of drug release for both compositions in the first half hour. Steady state release was achieved between the first 1 to 5 hours and maintained between 5 and 168 hours. During the 1 week trial, compositions released 0.68 mg and 0.69 mg of Ketorolac tromethamine. The percent cumulative drug release for Ket/EVA compositions was 0.95% and 0.98%.

We claim:

1. An implantable composition comprising:
one or more active ingredients for pain management, wherein the active ingredients are disposed in one or more layers of a two core coaxial implant;
a drug delivery matrix for storing one or more active ingredients within each core of the implant, the drug delivery matrix having a network of channels for releasing one or more active ingredients through pores open to the surface of the matrix, the drug delivery matrix comprising a biocompatible polymer; and
a coating applied to the external most surface of the implant, the coating comprising an erodible polymer that readily degrades in vivo, the erodible polymer for preventing an initial burst of active ingredient release.

2. The implantable composition of claim 1, wherein the coating degrades within 1-5 hours.

3. The implantable composition of claim 1, where the drug delivery matrix comprises a biocompatible and non-degradable or non-erodible polymer selected from the group comprising: ethylene vinyl acetate copolymer or others such as silicone, hydrogels such as crosslinked poly(vinyl alcohol) and poly(hydroxy ethylmethacrylate), acyl substituted cellulose acetates and alkyl derivatives thereof, partially and completely hydrolyzed alkylene-vinyl acetate copolymers, unplasticized polyvinyl chloride, crosslinked homo- and copolymers of polyvinyl acetate, crosslinked polyesters of acrylic acid and/or methacrylic acid, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, and chlorosulphonated polyolefines, collagan and combinations thereof.

4. The implantable composition of claim 1, wherein the one or more active ingredients are selected from a class of drugs selected from the group comprising: NSAIDS, steroidal active ingredients, DMARDS, opioids, α-2 adrenergic agonists, TCA's, SS(N)RI's, bisphosphonates, PSGAG's, and combinations thereof.

5. The implantable composition of claim 1, wherein the one or more active ingredients are selected from the group comprising: Carprofen, Clomazepam, Diazepam, Gabapentin, Amantadine, Tramadol, Meloxicam, Morphine, Fentanyl, Butorphanol, Toradol, Ibuprofen, Butorphanol and Carprofen, Tramadol and Lorazepam, Tramadol and Clonazepam, Tramadol with Meloxicam, Tramadol with Paracetamol, Tramadol and Clonazepam, Tramadol with Lorazepam, Tramadol and Diazepam, Tramadol and Gabapentin, Carprofen and Gabapentin, Carprofen and Clonazepam, Carprofen and Lorazepam, Carprofen and Amantadine, Morphine and Ibuprofen, Morphine and Carprofen, Morphine and Tramadol, Morphine and Amantadine, Fentanyl and Ibuprofen, Fentanyl and Tramadol, fentanyl and Carprofen, Fentanyl and Amantadine, Tramadol, Carprofen and Gabapentin, Tramadol, Carprofen and Clonazepam, Tramadol, Carprofen and Lorazepam, Diclofenac salt and Tramadol, and Gabapentin, and combinations thereof.

6. The composition of claim 5, wherein the diclofenac salt is selected from the group comprising sodium, potassium, or calcium.

7. The implantable composition of claim 1, wherein the coating comprises a lipid excipient.

8. The implantable composition of claim 6, wherein the lipid excipient comprises a blend of behenic acid esters with glycerol.

9. The implantable composition of claim 1, wherein the drug delivery matrix comprises ethylene vinyl acetate copolymer.

10. The implantable composition of claim 1, wherein the ethylene vinyl acetate copolymer is between 10 and 40% vinyl acetate.

11. The implantable composition of claim 1, wherein the drug delivery matrix provides a supervision-free, prolonged and sustained delivery of one or more active ingredients at desired therapeutic levels to ensure comfort, reduce stress, relieve pain, and treat physiological conditions in animals.

12. The implantable composition of claim 1, wherein the composition minimizes frequency of dosing and eliminates periodic dosing in the treatment of animals.

13. The implantable composition of claim 1, wherein the composition provides a drug delivery mechanism that circumvents the gastrointestinal tract, thereby minimizing or eliminating GI distress and ulcers.

14. The implantable composition of claim 1, wherein the composition minimizes or eliminates hepatotoxicity, and can be used to medicate older animals or animals with impaired liver function.

15. The implantable composition of claim 1, wherein the composition minimizes or eliminates renal toxicity, and can be used to medicate older animals or animals with impaired kidney function.

16. The implantable composition of claim 1, wherein the composition ensures safe supply of opioid pain medicines and minimizes abuse potential by pet owners and drug abusers.

17. The implantable composition of claim 1, wherein the composition eliminates administration of opioids and other controlled substance pain medicines by a licensed veterinarian in their place of practice or office.

18. The implantable composition of claim 1, wherein the shape of the composition is customizable to suit a particular condition or drug release conditions.

19. An implantable composition comprising:
one or more active ingredients for pain management, wherein the active ingredients are disposed in one or more layers of a two core coaxial implant; and
a drug delivery matrix for storing one or more active ingredients within each core of the implant, the drug delivery matrix having a network of channels for releasing one or more active ingredients through pores open to the surface of the matrix, the drug delivery matrix comprising a biocompatible polymer.

20. An implantable composition comprising:
two or more active ingredients for pain management, wherein the active ingredients are disposed in one or more layers of a two core coaxial implant;
two or more drug delivery matrices for storing one or more active ingredients within each core of the implant, at least one drug delivery matrix having a network of channels for releasing one or more active ingredients through pores open to the surface of the matrix, the drug delivery matrices comprising a biocompatible polymer; and
a coating applied to the external most surface of the implant, the coating comprising an erodible polymer that readily degrades in vivo, the erodible polymer for preventing an initial burst of active ingredient release.
